(19) United States
(12) Patent Application Publication
(10) Pub. No.: US 2007/0264726 A1
(43) Pub. Date: Nov. 15, 2007

(54) Microchannel array component, microchannel array for recovering biomolecules, and method for recovering biomolecules

(75) Inventor: Osamu Kogi, Tokyo (JP)

Correspondence Address: Reed Smith LLP;Suite 1400, 3110 Fairview Park Drive, Falls Church, VA, 22042-4503, US

(73) Assignee: Hitachi Software Engineering Co., Ltd.

(21) Appl. No.: 11/819,558

(22) Filed: Jun. 28, 2007

Related U.S. Application Data
(62) Division of application No. 10/939,484, filed on Sep. 14, 2004.

(30) Foreign Application Priority Data
Nov. 27, 2003 (JP) 2003-396915

(51) Int. Cl. G01N 33/00

(52) U.S. Cl. 436/501

(57) ABSTRACT

Various types of biomolecules as the objects of recovery included in a sample solution in minute amounts are separated in accordance with their affinity for various types of probe molecules and readily recovered. A sample solution 8 including biomolecules 7 as the objects of recovery are poured into a microchannel array 6. After the biomolecules 7 are bound to probe molecules which are fixed on the wall of a microchannel in the form of spots and which have a high affinity for the biomolecules 7, a second substrate having periodic protrusions is removed from the microchannel array 6. A portion of the microchannel array 6 that includes a spot in which the probe molecules that are bound to the biomolecules 7, which are the objects of recovery for the first substrate, are fixed is cut with a sharp cutting knife, for example, thereby obtaining a piece that retains on the surface the probe molecules that are bound to the biomolecules 7.

### CROSS-REFERENCE TO RELATED APPLICATION

This application is a Divisional application of U.S. application Ser. No. 10/939,484 filed Sep. 14, 2004. Priority is claimed based on U.S. application Ser. No. 10/939,484 filed Sep. 14, 2004, which claims the priority of Japanese Patent App. No. 2003-396915 filed on Nov. 27, 2003, the content of which is hereby incorporated by reference into this application.

### BACKGROUND OF THE INVENTION

1. Field of the Invention

The present invention relates to an array for recovering biomolecules contained in sample solutions in minute amounts and to a method for recovering biomolecules using such a recovering array.

2. Background Art

As an effective method for examining the expression state of genes, DNA probe arrays or DNA chips have begun to be used, in which many DNA probes divided by type are fixed on the surface of a solid body. JP Patent Publication (Kokai) No. 11-243997 A (1999) discloses an invention of a probe array. In the probe array, particles (probe particles) on which various types of probes are fixed are arranged in a certain order. Specifically, a plurality of tubules or grooves filled with various types of probe particles are arranged in parallel, and then, by injecting each single particle from each tubule or groove into another tubule or groove, a probe array is prepared in which various types of probe particles are constantly arranged in a certain order. By using the probe array, various types of probes are bound to particles having different particle sizes, and various types of fluorescence-labeled DNA are simultaneously measured.

By the technology described in JP Patent Publication (Kokai) No. 11-243997 A (1999), a capillary bead array is manufactured in which various types of probe molecules are fixed on the surface of beads that are arranged in a channel such as a glass capillary in a desired order. And then a sample solution including biomolecules that are the objects of analysis is poured into the capillary bead array. By binding the biomolecules as the objects of analysis to the probe molecules fixed on the surface of beads and having a high affinity for such biomolecules, the biomolecules as the objects of analysis can be fixed on the surface of the beads and can then be analyzed. However, using the capillary bead array of this type, it is significantly difficult to selectively remove beads on which the biomolecules as the objects of analysis are fixed. Consequently, it is significantly difficult to selectively recover such biomolecules. Therefore, a technology is required by which biomolecules as the objects of analysis that are included in sample solutions in minute amounts can be selectively recovered.

### SUMMARY OF THE INVENTION

It is an object of the present invention to provide an array and a method for effectively separating and recovering biomolecules as objects of analysis that are included in sample solutions in minute amounts depending on affinity for various probe molecules.

The inventor of the present invention, as a result of a dedicated research, found that the aforementioned object could be achieved by a microchannel array component made of a specific material and having microchannels, a microchannel array manufactured by combining the array component with a substrate of a specific structure, and a method for recovering biomolecules using the microchannel array, thereby arriving at the present invention.

In a first aspect, the present invention provides a microchannel array component. As a first substrate, a soft material such as soft resin, for example, which can be readily cut with a sharp tool, is selected, and a microchannel is prepared on the first substrate. On an undersurface and/or a wall surface of the microchannel, various types of probe molecules are fixed in the form of spots by chemical bonding in a desired sequence. By selecting a material such as soft resin, a biomolecule bound to a probe molecule in each specific position can be cut, separated, and recovered, so that the biomolecule can be used for inspection and analysis afterwards.

In a second aspect, the present invention provides a microchannel array for recovering biomolecules, which is manufactured by combining the aforementioned microchannel array component with a second substrate. The microchannel array component comprises the microchannel prepared on the first substrate, which is made of a soft material such as soft resin that can be readily cut with a sharp tool, and various types of probe molecules fixed on an undersurface and/or a wall surface of the microchannel in the form of spots by chemical bonding in a desired sequence. The second substrate comprises periodic protrusions of such dimensions that they can be fitted in the microchannel of the microchannel array component. The microchannel array for recovering biomolecules has the spots in which various types of probe molecules are fixed in the microchannel, and periodic protrusions. By disposing the periodic protrusions in the microchannel, a sample solution including biomolecules poured into the microchannel can be stirred, causing turbulence, thereby improving the efficiency of binding to the probe molecules. Moreover, by combining the array component with the second substrate, the structure of the array for recovering biomolecules can be simplified, so that the manufacturing costs can be reduced.

The form of protrusions disposed in the microchannel is not particularly limited. Cylindrical, rectangular columnar, conical, plate-like or corrugated walls or top surfaces, for example, may be used such that they can stir the sample solution poured into the microchannel as the protrusions obstruct the flow of the sample solution, and causing turbulence.

In a third aspect, the present invention provides a method for recovering biomolecules using the aforementioned microchannel array having the spots in which various types of biomolecules are fixed and the periodic protrusions. The sample solution including the biomolecules as the objects of recovery is poured into the microchannel of the microchannel array. By means of the periodic protrusions disposed in the microchannel, the sample solution is efficiently stirred while causing turbulence, and the biomolecules as the objects of recovery included in the sample solution are promptly bound to the probe molecules that are fixed in an undersurface and/or a wall surface of the microchannel in the form of spots and that have a high affinity for such biomolecule. By the periodic protrusions disposed in the microchannel, the sample solution including the biomolecules poured into the microchannel can be stirred, causing turbulence, thereby improving the efficiency of binding to the probe molecules.

In a fourth aspect, the aforementioned method for recovering biomolecules comprises a subsequent treatment process. The biomolecules as the objects of recovery included in the sample solution are fixed on an undersurface and/or a wall surface of the microchannel in the form of spots, and then the biomolecules are promptly bound to the probe molecules, which have a high affinity for such biomolecules. Thereafter, the substrate having the periodic protrusions is separated from the microchannel array component, and then, in a microchannel of the microchannel array component, a portion in which the probe molecules that are bound to the biomolecules as the objects of recovery are fixed is cut, using a tool such as a sharp cutting knife. The cut piece in which probe molecules are fixed can be subjected to fluorescent measurement, using a flow meter, for example, in the same manner as that used for conventional beads. In this case, since the cutting order is specified, there is no need for arrangement in a fixed order or sequence, as in the case of beads. In addition, according to the present invention, it is easy to cut only a desired portion for measurement.

In a fifth aspect, in the method for recovering biomolecules according to the fourth aspect, a portion of a microchannel of the microchannel array component in which the probe molecules that are bound to the biomolecules as the objects of recovery are fixed is cut, using a tool such as a sharp cutting knife. By chemically treating the piece cut from the substrate so as to cut the bonding between the probe molecules fixed on the surface of the piece and the biomolecules as the objects of recovery, the biomolecules are recovered. Thus, only a specific type of biomolecules can be efficiently recovered.

According to the present invention, biopolymers included in a sample solution in minute amounts can be readily and reliably separated in accordance with affinity for various types of probe molecules, and can be recovered. Namely, a significantly effective tool can be provided for users who are engaged in research and development in life sciences.

### BRIEF DESCRIPTION OF THE DRAWINGS

FIG. 1 schematically shows a method of manufacturing a microchannel array provided with spots in which various types of probe molecules are fixed, and periodic protrusions.

FIG. 2 schematically shows a method of manufacturing a microchannel array provided with spots in which various types of probe molecules are fixed, and periodic protrusions.

FIG. 3 schematically shows a method of manufacturing a microchannel array provided with spots in which various types of probe molecules are fixed, and periodic protrusions.

FIG. 4 schematically shows a microchannel array provided with spots in which various types of probe molecules are fixed, and periodic protrusions.

FIG. 5 schematically shows a sample solution including biomolecules as the objects of recovery being poured into a microchannel array of the present invention.

FIG. 6 schematically shows a method by which probe molecules and biomolecules are recovered from a microchannel array.

FIG. 7 schematically shows a method by which probe molecules and biomolecules are recovered from a microchannel array.

FIG. 8 schematically shows an operation by which biomolecules as the objects of recovery are recovered from a small piece that is cut from a microchannel array.

### DESCRIPTION OF THE PREFERRED EMBODIMENTS

An embodiment of the present invention is described in detail below with reference to the drawings.

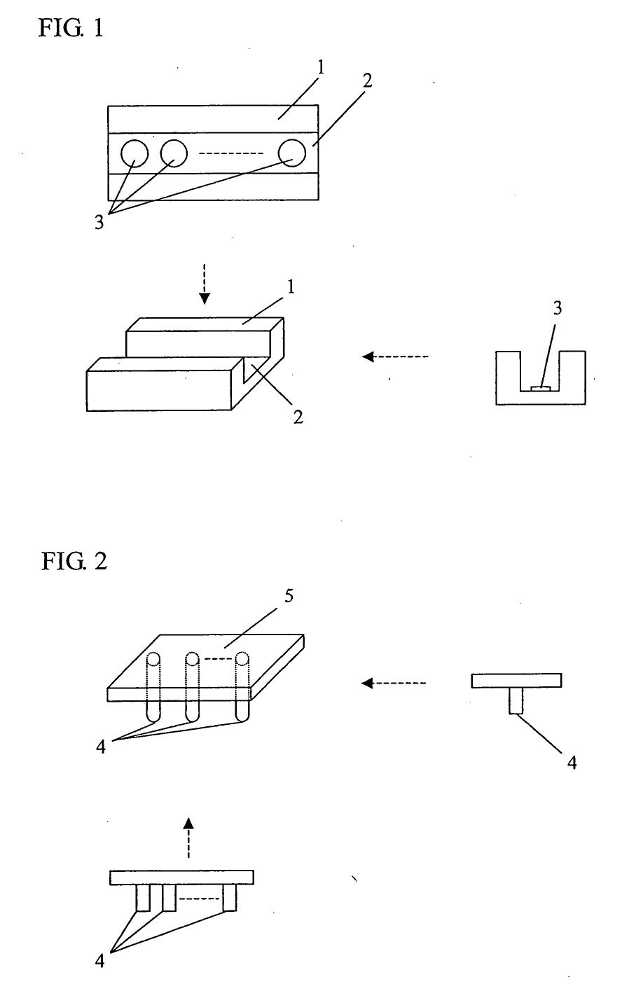

FIGS. 1 to 3 schematically show a method of manufacturing a channel structure provided with spots in which various types of probe molecules are fixed, and periodic protrusions. FIG. 4 schematically shows a microchannel array provided with spots in which various types of probe molecules are fixed, and periodic protrusions.

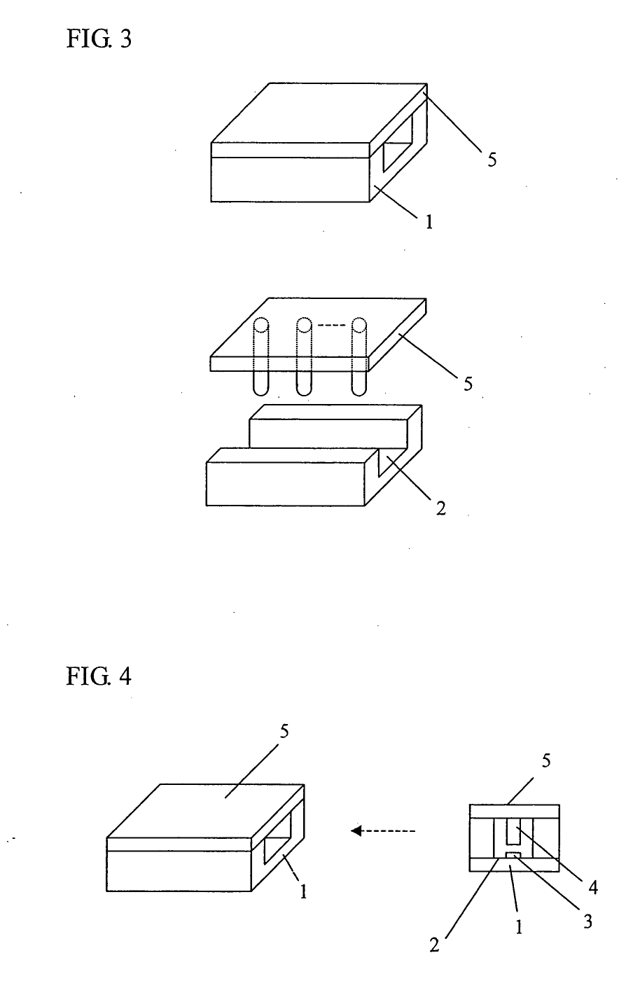

FIG. 1 shows a perspective, a plan, and a lateral view of a first substrate 1. A microchannel 2 is prepared on the first substrate 1, which is made of a material such as soft resin, which can be readily cut with a sharp tool. On an undersurface and/or a wall surface of the microchannel (the undersurface in the example of FIG. 1), spots 3 are prepared, in which various types of probe molecules are fixed by chemical bonding in a desired sequence. FIG. 2 shows a perspective, a front, and a lateral view of a second substrate 5 provided with periodic protrusions 4. FIG. 3 shows perspective views of the second substrate 5 having been fitted in the microchannel 2 of the first substrate 1, and the manner of fitting. FIG. 4 shows a perspective view in which the second substrate 5 is fitted in the microchannel 2 of the first substrate 1, and a cross-section view. By combining the microchannel 2 of the first substrate 1 with the substrate 5 of such dimensions that it can be fitted in the microchannel 2, the substrate 5 having the periodic protrusions 4 (cylindrical protrusions in FIGS. 2 and 3), a microchannel array is manufactured that has the spots 3 in which various types of probe molecules are fixed, and periodic protrusions 4. The positional relationship of the spots 3 the periodic protrusions 4 is not especially limited. They may or may not be disposed in a corresponding manner.

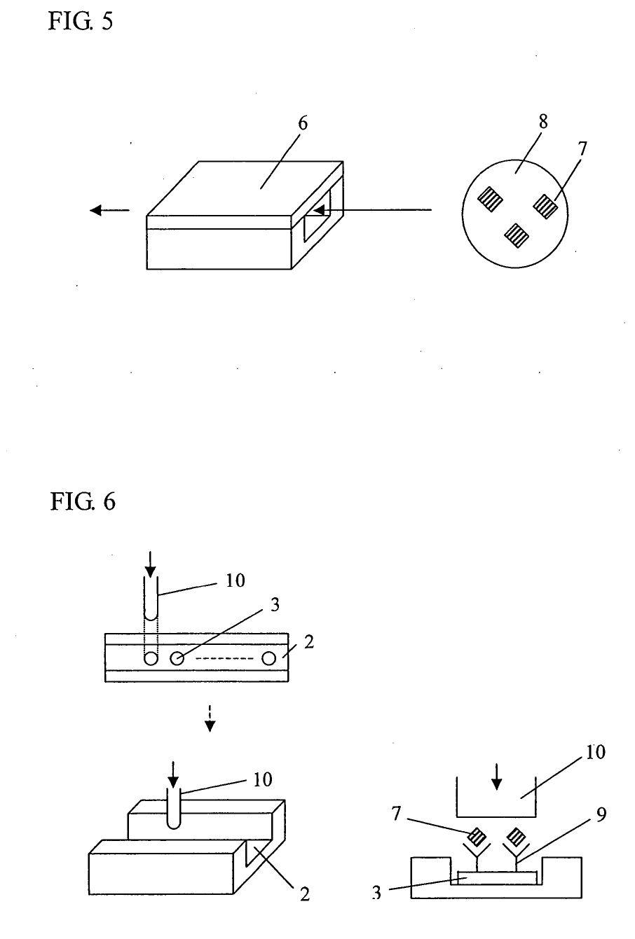

FIG. 5 schematically shows how a sample solution 8 including biomolecules as the objects of recovery is poured into the microchannel array 6. The sample solution 8 including biomolecules 7 as the objects of recovery is poured into the microchannel array 6 explained in FIGS. 1 to 4. The sample solution 8 is efficiently stirred by the periodic protrusions in the microchannel of the microchannel array 6 such that the biomolecules 7 as the objects of recovery included in the sample solution 8 are promptly bound to the probe molecules that are fixed on the wall surfaces of the microchannel in spots and that have a high affinity for the biomolecules 7.

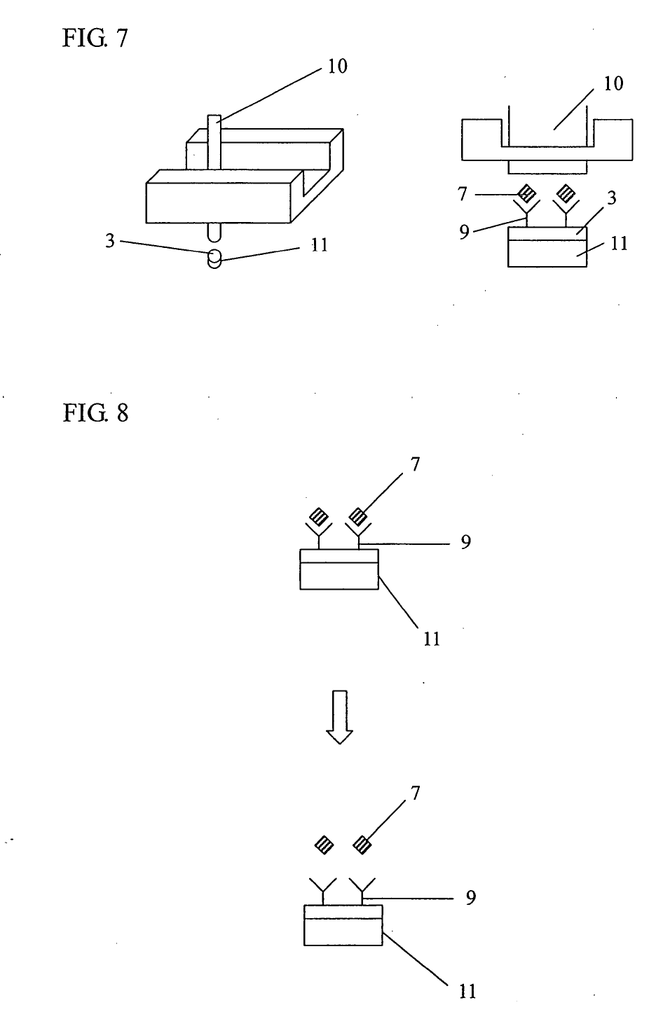

FIGS. 6 and 7 schematically show perspective, plan, and cross-sectional views of a method by which the probe molecules and the biomolecules as the objects of recovery are recovered from the microchannel array. An aqueous solution including the biomolecules 7 as the objects of recovery is poured into the microchannel array. After the biomolecules 7 are bound to the probe molecules 9 that are fixed on the walls surface of the microchannel in spots and that have a high affinity for the biomolecules 7, the second substrate provided with the periodic protrusions is removed from the microchannel array. A portion of the microchannel 2 in the microchannel array that includes the spot 3 at which the probe molecules 9 that are bound to the biomolecules 7 as the objects of recovery for the first substrate are fixed, is cut with a sharp cutting knife 10, thereby obtaining a small piece 11 that retains on the surface the probe molecules 9 to which the biomolecules 7 are bound. The thus cut-out piece 11, as shown in FIG. 7, may be taken out by penetrating through the first substrate or may be removed from the cutting knife 10 to which the cut piece 11 has adhered.

FIG. 8 schematically shows an operation by which biomolecules as the objects of recovery are recovered from the small piece that is cut from the microchannel array. In FIG. 8, the piece 11 that is cut from the channel structure retains the probe molecules 9 on the surface thereof, and the biomolecules 7 as the objects of recovery are bound to the probe molecules 9. The cut piece 11 is then chemically treated so as to cut the bonding between the probe molecules 9 and the biomolecules 7 as the objects of recovery, thereby recovering the biomolecules 7.

According to the present invention, a biopolymer included in a sample solution in minute amounts can be readily and reliably separated in accordance with affinity for various types of probe molecules, and then be recovered. Namely, the invention provides an effective tool for users who are engaged in research and development in life sciences. The present invention can therefore contribute to the progress of life sciences, and the development of medical diagnosis and medicines.

What is claimed is:

1. A method for recovering biomolecules comprising:
delivering a sample solution including biomolecules as the objects of recovery to the microchannel of a microchannel array, a microchannel array having spots in which various types of probe molecules are fixed and periodic protrusions; and
promptly binding the biomolecules as the objects of recovery included in said sample solution to probe molecules that are fixed in the undersurface and/or the wall surface of said microchannel in the form of spots and that have a high affinity for said biomolecules;
wherein said microchannel array comprises
a microchannel array component comprising a first substrate made of a soft resin material on which a microchannel is disposed, wherein various types of probe molecules are fixed by chemical bonding on an undersurface and/or a wall surface of said microchannel in a desired sequence in the form of spots; and
a second substrate combined with said microchannel array component, the second substrate having periodic protrusions of such dimensions that they can be fitted in the microchannel of said microchannel array component.

2. The method for recovering biomolecules according to claim 1, comprising, following the step of binding the biomolecules as the objects of recovery included in said sample solution promptly to the probe molecules that are fixed in the undersurface and/or the wall surface of the microchannel in the form of spots and that have a high affinity for said biomolecules, separating the substrate having periodic protrusions from the microchannel array component, and cutting out, using a sharp tool, a portion of the microchannel of said microchannel array component in which the probe molecules that are bound to the biomolecules as the objects of recovery are fixed.

3. The method for recovering biomolecules according to claim 1, comprising, following the step of binding the biomolecules as the objects of recovery included in said sample solution promptly to the probe molecules that are fixed in the undersurface and/or the wall surface of the microchannel in the form of spots and that have a high affinity for said biomolecules, separating the substrate having periodic protrusions from the microchannel array component, cutting out, using a sharp tool, a portion of the microchannel of said microchannel array component in which the probe molecules that are bound to the biomolecules as the objects of recovery are fixed, and recovering the biomolecules by chemically treating the piece cut from the substrate so as to cut the bonding between the probe molecules and the biomolecules as the objects of recovery.
